(19) United States
(12) Patent Application Publication
(10) Pub. No.: US 2025/0073165 A1
(43) Pub. Date: Mar. 6, 2025

(54) OLANZAPINE, COMPOSITIONS THEREOF AND METHODS OF USE THEREOF

(71) Applicant: Teva Pharmaceuticals International GmbH, Jona (CH)

(72) Inventors: Dario KLARIC, Zapresic (HR); Dunja SILIC, Zagreb (HR); Antonia Giacobi MARTINELLI, Zagreb (HR); Sanja Matecic MUSANIC, Zagreb (HR); Damir ŠAHNIC, Zagreb (HR)

(21) Appl. No.: 18/730,347

(22) PCT Filed: Jan. 20, 2023
(86) PCT No.: PCT/IB2023/050494

§ 371 Date: Jul. 19, 2024

Related U.S. Application Data
(60) Provisional application No. 63/301,202, filed on Jan. 20, 2022.

(51) Int. Cl. A61K 9/14; A61K 31/5513

(52) U.S. Cl. CPC A61K 9/14; A61K 31/5513

(57) ABSTRACT

The disclosure is directed to olanzapine, compositions and methods of use thereof, wherein the olanzapine exhibits good flowability and syringeability.

### CROSS-REFERENCE TO RELATED APPLICATIONS

This application claims the benefit of priority of U.S. Provisional Patent Application No. 63/301,202, filed Jan. 20, 2022, the contents of which are incorporated herein by reference in their entirety for all purposes.

### FIELD

Provided herein is olanzapine drug substance having favorable flow characteristics and drug product comprising same.

### BACKGROUND OF THE DISCLOSURE

Olanzapine is a well characterized and commonly prescribed atypical antipsychotic drug available in oral and parenteral (intramuscular, IM) formulations. Olanzapine belongs to the thienobenzodiazepine class and its chemical name is 2-methyl-4-(4-methyl-1-piperazinyl)-10H-thieno[2,3-b][1,5]benzodiazepine.

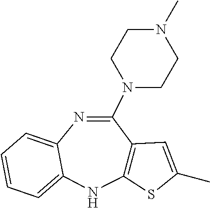 [SMILES: Cc1cc2c(s1)Nc1ccccc1N=C2N1CCN(C)CC1]

U.S. Pat. Nos. 5,229,382 and 5,736,541 describe olanzapine Form I and Form II, respectively. U.S. Pat. No. 7,323,459 describes olanzapine crystalline Forms H, G, Y, X, K, S, Q, Z, and J. As a free base or salt, e.g. hydrochloride or pamoate, olanzapine is an active pharmaceutical ingredient (API) of medications used in the treatment of schizophrenia and other neuropsychiatric diseases and disorders. Oral formulations of olanzapine (ZYPREXA) and a long acting intramuscular (IM) depot preparation (ZYPREXA RELPREVV®, Eli Lilly) containing olanzapine pamoate are approved in the U.S. for the treatment of adults and adolescents affected by schizophrenia. The oral formulations of olanzapine are also approved in the U.S. for the treatment of bipolar I disorder. An IM formulation of olanzapine is approved for the treatment of adults with acute agitation associated with schizophrenia or bipolar I mania.

Olanzapine exhibits poor water solubility, dissolution and flow properties. Methods to improve these characteristics may include, for example, particle size reduction, salt screening, spray drying and encapsulating in microspheres. However, not all known methods are practical for all API presentations. A reduction in API particle size may increase solubility but may detrimentally affect flowability and filling. For example, for olanzapine presented in a pharmaceutical container, i.e. vial, syringe, capsule, bottle, sachet or ampoule for parenteral administration with an excipient, flowability of the API is critical for dose accuracy and container filling (e.g. vialing). Furthermore, when combined with the excipient, one or more of favorable dissolution or suspension and successful syringeability properties are required.

Thus, there exists the need for a flowable formulation of olanzapine that exhibits those properties.

### SUMMARY

Olanzapine is difficult to vial due to its inherent cohesiveness. An unexpected correlation between flow function, low relative standard deviation (RSD) of fill weight and good filling speed and certain ranges of D(90) particle size distribution (PSD), D(3,2) PSD, tapped density or any combination thereof of the olanzapine drug substance has been identified. The inventors of the present disclosure have identified features of olanzapine drug substance having favorable flow characteristics that provide for dose accuracy and vialing with concomitant good suspension and syringeability properties for an olanzapine parenteral drug product. Disclosed herein are olanzapine drug substance, and a container or kit comprising the olanzapine having the desirable characteristics, as well as to pharmaceutical compositions and methods of use thereof.

In one aspect, provided herein is olanzapine or a pharmaceutically acceptable salt thereof having at least one of the following properties: a particle size distribution characterized by a D(90) of from about 20 to about 37 μm, or a particle size distribution characterized by a D(3,2) of from about 5.5 to about 7.5 μm, or a tapped density from about 0.35 to 0.44 g/ml; or any combination thereof. The olanzapine or a pharmaceutically acceptable salt thereof exhibits a flow function of about 2.0 or less and/or an RSD of filling weight (%) of about 1.5% or less and/or a filling speed of 14% or higher. In some embodiments, the olanzapine or a pharmaceutically acceptable salt thereof exhibits a flow function of about 2.0 or less or from about 1.0 to about 2.0, or from 1.4 to about 2.0. In some embodiments, the olanzapine or a pharmaceutically acceptable salt thereof exhibits an RSD of filling weight (%) of about 1.5% or less or about from 1.0% to about 1.5%. In some embodiments, the olanzapine or a pharmaceutically acceptable salt thereof exhibits a filling speed of 14% or higher or about 14% to about 20%. In some embodiments the olanzapine is olanzapine base. In some embodiments the olanzapine is an olanzapine salt. In some embodiments the olanzapine is olanzapine polymorph. In some embodiments the olanzapine is olanzapine Form II.

Further provided is a container comprising the olanzapine Form II having a particle size distribution characterized by a D(90) of from about 20 to about 37 μm, a particle size distribution characterized by a D(3,2) of from about 5.5 to about 7.5 μm, a tapped density from about 0.35 to 0.44 g/ml; or any combination thereof. The olanzapine exhibits a flow function of about 2.0 or less and/or an RSD of filling weight (%) of about 1.5% or less and/or a filling speed of 14% or higher. In some embodiments the olanzapine is olanzapine base. In some embodiments the olanzapine is an olanzapine salt. In some embodiments the olanzapine is olanzapine polymorph. In some embodiments the olanzapine is olanzapine Form II. In some embodiments, the container comprises sterilized or aseptic olanzapine Form II. The container may be a vial or other pharmaceutically acceptable vessel.

In another aspect, a pharmaceutical composition comprises olanzapine having at least one of the following properties: a particle size distribution characterized by a D(90) of from about 20 to about 37 μm, a particle size distribution characterized by a D(3,2) of from about 5.5 to about 7.5 μm, a tapped density from about 0.35 to 0.44 g/ml; or any combination thereof; and one or more pharmaceutically acceptable excipient. In some embodiments the olanzapine is olanzapine Form II. In some embodiments, the olanzapine particle size distribution is characterized by a D(90) of from 20 to about 37 μm. The olanzapine exhibits a flow function of about 2.0 or less and/or an RSD of filling weight (%) of about 1.5% or less and/or a filling speed of 14% or higher. In some embodiments, the olanzapine or a pharmaceutically acceptable salt thereof exhibits a flow function of about 2.0 or less or from about 1.0 to about 2.0, or from 1.4 to about 2.0. In some embodiments, the olanzapine or a pharmaceutically acceptable salt thereof exhibits an RSD of filling weight (%) of about 1.5% or less or about from 1.0% to about 1.5%. In some embodiments, the olanzapine or a pharmaceutically acceptable salt thereof exhibits a filling speed of 14% or higher or about 15% to about 20%. In some embodiments the olanzapine is olanzapine base. In some embodiments the olanzapine is an olanzapine salt. In some embodiments the olanzapine is olanzapine polymorph. In some embodiments the olanzapine is olanzapine Form II. In some embodiments, the pharmaceutical composition is in the form of a suspension or solution for parenteral administration and comprises for example one or more polymer or non-polymer excipient. The pharmaceutical composition may be in the form of, for example, a subcutaneous or intramuscular injectable preparation, for example housed in a syringe. The syringe may include about 100 to about 1,000 mg olanzapine, or about 300 to about 800 mg olanzapine in a polymer or non-polymer excipient.

Further provided is a kit comprising olanzapine having at least one of the following properties: a particle size distribution characterized by a D(90) of from about 20 to about 37 μm, a particle size distribution characterized by a D(3,2) of from about 5.5 to about 7.5 μm, a tapped density from about 0.35 to 0.44 g/ml; or any combination thereof; optionally further comprising a pharmaceutically acceptable excipient, a syringe and or instructions for use. The olanzapine exhibits a flow function of about 2.0 or less and/or an RSD of filling weight (%) of about 1.5% or less and/or a filling speed of 14% or higher. In some embodiments the olanzapine is olanzapine base. In some embodiments the olanzapine is an olanzapine salt. In some embodiments the olanzapine is olanzapine polymorph. In some embodiments the olanzapine is olanzapine Form II. The olanzapine may be in a container, for example a vial, capsule, bottle, sachet, syringe or ampoule.

Further provided is a method of treating a subject suffering from a neuropsychiatric disease or otherwise in need of the treatment, comprising administering to the subject olanzapine having at least one of the following properties: a particle size distribution characterized by a D(90) of from about 20 to about 37 μm, a particle size distribution characterized by a D(3,2) of from about 5.5 to about 7.5 μm, a tapped density from about 0.35 to 0.44 g/ml; or any combination thereof; optionally further comprising a pharmaceutically acceptable excipient, a syringe and or instructions for use. The olanzapine exhibits a flow function of about 2.0 or less and/or an RSD of filling weight (%) of about 1.5% or less and/or a filling speed of 14% or higher. In some embodiments the olanzapine is olanzapine base. In some embodiments the olanzapine is an olanzapine salt. In some embodiments the olanzapine is olanzapine polymorph. In some embodiments the olanzapine is olanzapine Form II. Preferably, the olanzapine provides a therapeutically effective plasma concentration of olanzapine for a period of at least about 21 days after administration to the subject.

### BRIEF DESCRIPTION OF THE FIGURES

FIG. 1 is an X-ray diffraction analysis (XRD) profile of olanzapine Form II.

FIG. 2 is an electron microscopy photograph (×1,000) showing particle morphology of micronized olanzapine, form II.

FIGS. 3A-3L provide bivariate plots showing the correlation between flow function and D(90), D(3,2) and TD (FIGS. 3A, 3B and 3C, respectively); RSD of fill weight and D(90), D(3,2), TD and flow function (FIGS. 3D, 3E, 3F, 3G, respectively) and filling speed (%) and D(90), D(3,2), TD and flow function (FIGS. 3H, 3J, 3K, 3L, respectively).

FIGS. 4A-4C are graphs depicting PSD of bulk API compared to PSD of API in vial after sterilization. The Y axes represent volume density (%, linear scale) and X axes represent size in micrometers (μ, log scale).

### DETAILED DESCRIPTION OF THE DISCLOSURE

Reference will now be made to the exemplary embodiments and specific language will be used herein to describe the same. It will nevertheless be understood that no limitation of the scope of the invention is thereby intended. Alterations and further modifications of the inventive features illustrated herein, and additional applications of the principles of the inventions as illustrated herein, which would occur to one skilled in the relevant art and having possession of this disclosure, are to be considered within the scope of the invention.

The present disclosure provides olanzapine, or pharmaceutically acceptable salt thereof, having a particle size distribution characterized by a D(90) of from about 20 μm to about 37 μm, a particle size distribution characterized by a D(3,2) of from about 5.5 μm to about 7.5 μm, or a tapped density from about 0.35 g/ml to 0.44 g/ml, or any combination thereof. In some embodiments, the particle size distribution is characterized by a D(90) of from 20 μm to about 37 μm. In some embodiments, the particle size distribution is characterized by a D(90) of from about 24 μm to about 35 μm. The particle size distribution of the olanzapine may be characterized by a D (3,2) of from about 5.5 μm to about 7.5 pm. In some embodiments, the particle size distribution is characterized by a D(3,2) of from about 5.8 μm to about 7.0 μm. The tapped density of the olanzapine is from about 0.35 g/ml to about 0.44 g/ml.

In some embodiments, the olanzapine has a particle size distribution characterized by a D(90) of from about 20 μm to about 37 μm and by a D(3,2) of from about 5.5 μm to about 7.5 μm.

In some embodiments, the olanzapine has a particle size distribution characterized by a D(90) of from about 20 μm to about 37 μm and by a tapped density of about 0.35 g/ml to about 0.44 g/ml.

In some embodiments, the olanzapine has a particle size distribution characterized by a D(3,2) of from about 5.5 μm to about 7.5 μm and by a tapped density of about 0.35 g/ml to about 0.44 g/ml.

In alternative embodiments, the olanzapine has a particle size distribution characterized by a D(90) of from about 20 μm to about 37 μm, by a D(3,2) of from about 5.5 μm to about 7.5 μm and by a tapped density of about 0.35 g/ml to about 0.44 g/ml.

The olanzapine drug substance disclosed herein has a flow function, as measured by a FT4 Powder Rheometer, of about 2.0 or less. In some embodiments, the flow function, as measured by a FT4 Powder Rheometer, is about 1.0 to about 2.0.

The olanzapine drug substance disclosed herein results in a RSD % of fill weight of 1.5% or less, or from 1.0% to 1.5%. In some embodiments, the filling speed is 14% or higher or from 14% to 20%.

Further provided is a container comprising the olanzapine disclosed herein, for example olanzapine, or pharmaceutically acceptable salt thereof, having a particle size distribution characterized by a D(90) of from about 20 μm to about 37 μm, a particle size distribution characterized by a D(3,2) of from about 5.5 μm to about 7.5 μm, or a tapped density from about 0.35 g/ml to 0.44 g/ml, or any combination thereof. In some embodiments the container is a vial, a syringe or an ampoule. In specific embodiments, the container is a vial. The container may comprise about 100 mg to about 1,000 mg olanzapine, or about 300 mg to about 700 mg olanzapine. In some embodiments, the olanzapine is terminally sterilized in the container. In yet other embodiments, the olanzapine is aseptic.

Further provided is a pharmaceutical composition comprising the olanzapine disclosed herein and one or more pharmaceutically acceptable excipient. The olanzapine or pharmaceutically acceptable salt thereof, having a particle size distribution characterized by a D(90) of from about 20 μm to about 37 μm, a particle size distribution characterized by a D(3,2) of from about 5.5 μm to about 7.5 μm, or a tapped density from about 0.35 g/ml to 0.44 g/ml, or any combination thereof. The pharmaceutical composition maybe in the form of a solution or suspension for parenteral administration. In some embodiments, the pharmaceutical composition is an aqueous suspension or a non-aqueous suspension. The pharmaceutical composition may include a pharmaceutically acceptable excipient which comprises a polymer or non-polymer. In some embodiments, the excipient comprises a polymer, which may be a biodegradable polymer comprising poly(lactide), poly(glycolide), poly(lactide-co-glycolide), poly-1-lactic acid, poly-d-lactic acid, polyethylene glycol or a copolymer of the foregoing, poly(aliphatic carboxylic acids), copolyoxalates, polycaprolactone, polydioxanone, poly(ortho carbonates), poly(acetals), poly(lactic acid-caprolactone), polyorthoesters, poly(glycolic acid-caprolactone), poly(amino acid), polyesteramide, polyanhydrides, polyphosphazines, poly(alkylene alkylate), biodegradable polyurethane, polyvinylpyrrolidone, polyalkanoic acid, albumin, chitosan, casein, waxes or blends or copolymers thereof. In some embodiments, the pharmaceutical compositions further include a solvent.

In some embodiments, the pharmaceutical composition is in the form of an injectable preparation, for example a in a vial or in a syringe. The pharmaceutical composition comprises from about 100 mg to about 1,000 mg olanzapine, from about 300 mg to about 700 mg olanzapine, or from about 300 to about 600 mg olanzapine, or a pharmaceutically acceptable salt thereof. The pharmaceutical composition provides a therapeutically effective plasma concentration of olanzapine for the treatment of a neuropsychiatric disease for at least 14 days, or at least 21 days after administration to a patient. In some embodiments, the pharmaceutical composition provides a therapeutically effective plasma concentration of olanzapine for about 14 days, 21 days, about 28 days, about 30 days, about 42 days or about 56 days. The olanzapine or pharmaceutically acceptable salt thereof may be provided in a kit comprising the container and optionally further comprising a pharmaceutically acceptable excipient, a syringe and/or instructions for use.

Further provided is a method of treating a subject suffering from a neuropsychiatric disease or otherwise in need of the treatment, comprising administering to the subject the olanzapine or the pharmaceutical composition disclosed herein, wherein the olanzapine or the pharmaceutical composition provides a therapeutically effective plasma concentration of olanzapine for a period of at least about 14 days or at least 21 days or for about 14 days, about 21 days, about 28 days, about 30 days, about 42 days or about 56 days following administration to a patient. The pharmaceutical composition is administered to the subject parenterally, for example intramuscularly or subcutaneously, particularly subcutaneously. In some embodiments, the neuropsychiatric disease is schizophrenia or bipolar disorder.

Further provided is a process of filling a container with olanzapine or a pharmaceutically acceptable salt thereof, the process comprising providing olanzapine having a particle size distribution characterized by a D(90) of from about 20 μm to about 37 μm, a particle size distribution characterized by a D(3,2) of from about 5.5 μm to about 7.5 μm, or a tapped density from about 0.35 g/ml to 0.44 g/ml; or any combination thereof; and filling the container using a semi-automatic or fully automatic filling machine; thereby filling the container with the olanzapine or a pharmaceutically acceptable salt thereof.

Further provided is a process of filling a container with olanzapine or a pharmaceutically acceptable salt thereof, wherein the RSD % of the filling weight is less than 1.5% or the filling speed is 14% or higher, thereby filling the container with the olanzapine or a pharmaceutically acceptable salt thereof.

In some embodiments of the processes, the RSD % of fill weight is 1.5% or less, or from 1.0% to 1.5%. In some embodiments of the processes, the filling speed is 14% or higher or from 14% to 20%. In some embodiments of the processes, the container is a vial, syringe, capsule, bottle, sachet or ampoule, particularly a vial.

The relative standard deviation (RSD) is the ratio of standard deviation and mean of the sample. It is the coefficient of variation to describe accuracy of the filling process.

The terms D(90), D(99) and D(100) are well understood in the art. For example, a D(90) (or d(90) of 25 μm, means that 90% (by volume) of the particles have a diameter less than or equal to 25 μm. For example, a D(99) (or d(99)) of 50 μm, means that 99% (by volume) of the particles have a diameter less than or equal to 50 μm. For example, a D(100) of 300 μm, means that 100% (by volume) of the particles have a diameter less than or equal to 300 μm. D(3,2) refers to the surface area moment mean (Sauter Mean Diameter, SMD) and may be relevant where surface area is important e.g. dissolution and reflects the amount of fine particles in the sample. D(4,3) refers to the volume/mass moment mean (De Brouckere Mean Diameter) and reflects the size of particles which constitute the bulk of the sample. Particle size may be determined by means of laser diffractometry. In some embodiments, the particle size may be determined using a Mastersizer device from Malvern Instruments.

Tapped density (TD) refers to the ability of a powder sample to pack under taps and gives a measure of the powder cohesiveness which can be linked to its flowability and filling performance. Tapped density may be determined using a tapped density meter, as described in the Examples, infra.

Flow function (FF) is a parameter commonly used to rank flowability of powders, with higher values denoting better flow properties of a sample. The FF was obtained as described in the Examples infra. The olanzapine described herein is in the form of a cohesive powder, having favorable flowability properties, for example having a flow function value of about 2.0 or less, for example having a flow function of from 1.4 to 2.0 as measured, for example, using a FT4 Powder Rheometer. In some embodiments, the flow function of the olanzapine, as measured by a FT4 Powder Rheometer, is about 2.0 or less, or about 1.4 to 2.0.

Relative standard deviation of filling weight is calculated as follows:

$$RSD ( \% ) = ( S D ( mg ) ) / ( MEAN ( mg ) )$$

where RSD is relative standard deviation, SD is standard deviation of filling weight distribution in milligrams and MEAN is the average filling weight in milligrams.

In some embodiments, the RSD % of fill weight is 1.5% or less.

Filling speed as used herein refers to the number of units per minute as a percent (%) of the maximum capacity of the machine according to its technical specification. In some embodiments, the filling speed is 14% or higher. In some embodiments, the filling speed is 14% to 20%. In some embodiments, the filling speed is higher than 20%.

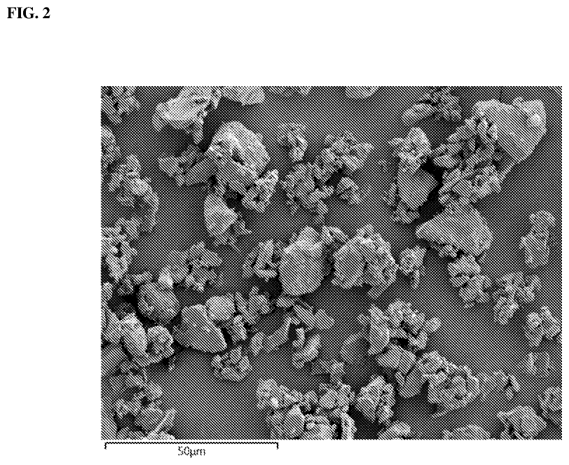

Particle morphology can be obtained by examining the particles, for example, in a scanning electron microscope (SEM). The particle morphology of the olanzapine disclosed herein is irregular and is substantially as shown in FIG. 2.

The singular forms “a,” “an,” and “the” may refer to plural articles unless specifically stated otherwise.

The term “about,” as used herein, is intended to qualify the numerical values which it modifies, denoting such a value as variable within a margin of ±10 wt %. The endpoints of all ranges directed to the same component or property are inclusive and independently combinable (e.g., ranges of “less than or equal to 25 wt %, or 5 wt % to 20 wt %,” is inclusive of the endpoints and all intermediate values of the ranges of “5 wt % to 25 wt %,” etc.).

As used herein the term “pharmaceutically acceptable” refers to those compounds, materials, compositions, and/or excipients which are, within the scope of sound medical judgment, suitable for contact with the tissues of human beings without excessive toxicity, irritation, allergic response, or other problem complications commensurate with a reasonable benefit/risk ratio.

As used herein, a vial refers to a container suitable for use in packaging, distributing, and using compositions. A vial may be single dose vial (i.e., a vial containing a quantity of API equal to a single dose, such as a single human dose). Alternatively, the vial may contain more than one dose (multi-dose vial). As used herein, vialing refers to placing an amount of an API, for example olanzapine, into a single vial or other container. Vialing may be performed manually, semi-automatically or automatically.

The pharmaceutical compositions of the disclosure include the active pharmaceutical ingredient, or a salt form thereof, and at least one pharmaceutically acceptable carrier or excipient. Said pharmaceutical compositions can be formulated using polymers and or other suitable materials using methods known in the art. The pharmaceutical compositions may be aqueous or non-aqueous. In some embodiments, the API, e.g. olanzapine, and the excipient, e.g. a polymer, form a depot in situ, for example, following intramuscular or subcutaneous administration, from which the API is released over time.

Examples of excipients useful for preparing the pharmaceutical compositions of the disclosure include biodegradable polymers including poly (lactide) (poly lactic acid, PLA), poly(glycolide) (poly glycolic acid, PGA), poly(lactide-co-glycolide) (PLGA), poly-1-lactic acid (PLLA), poly-d-lactic acid (PDLA), polyethylene glycol or copolymers of the foregoing, poly(aliphatic carboxylic acids), copolyoxalates, polycaprolactone, polydioxanone, poly(ortho carbonates), poly(acetals), poly(lactic acid-caprolactone), polyorthoesters, poly(glycolic acid-caprolactone), poly(amino acid), polyesteramide, polyanhydrides, polyphosphazines, poly(alkylene alkylate), biodegradable polyurethane, polyvinylpyrrolidone, polyalkanoic acid; albumin, chitosan, casein, gelatin, waxes or blends or copolymers thereof. Non-polymeric excipients include sucrose acetate isobutyrate (SAIB).

Methods for formulating the active ingredient into a pharmaceutical composition suitable for use in the instant methods are understood by those with skill in the art.

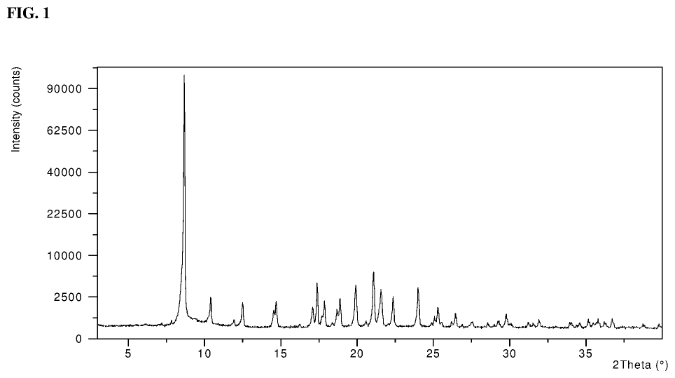

The olanzapine used in the methods of the disclosure can be present in the pharmaceutical compositions as either olanzapine or as a pharmaceutically acceptable salt of olanzapine. Examples of pharmaceutically acceptable salts include tartrate salt, such as a (D)(−) tartrate salt or a (L)(+) tartrate salt, a hydrochloride salt, a citrate salt, a malate salt, particularly a D-malate salt, a fumarate salt, a succinate salt, a benzoate salt, a benzenesulfonate salt, a pamoate salt, a formate salt, a malonate salt, a 1,5-naphthalenedisulfonate salt, a salicylate salt, a cyclohexanesulfamate salt, a lactate salt, a mandelate salt, particularly an (R)(−) mandelate salt, a glutarate salt, an adipate salt, a squarate salt, a vanillate salt, an oxaloacetate salt, an ascorbate salt, particularly an (L)-ascorbate salt and a sulfate salt. In an aspect, the form of olanzapine is olanzapine base Form II, having an XRD profile as shown in FIG. 1.

Parenteral administration refers to administration other than oral administration. As used herein, “subcutaneously administered” refers to administration into the layer of skin that is directly below the dermis and epidermis. The term specifically excludes intramuscular and intravenous methods of administration. In an aspect, methods of subcutaneous administration include subcutaneous injections.

As used herein, a “therapeutically effective dose” refers to the amount of olanzapine that is sufficient to alleviate the positive and/or negative symptoms of schizophrenia and/or bipolar disorder in the patient.

Each container comprises from about 100 mg to about 1,000 of olanzapine or a pharmaceutically acceptable salt of olanzapine. In some embodiments each container comprises from about 150 mg to about 800 mg of olanzapine or a pharmaceutically acceptable salt of olanzapine, or from about 300 to about 700 mg of olanzapine or a pharmaceutically acceptable salt of olanzapine. In some embodiments, the container is a vial, specifically a glass vial. In some embodiments, the containers comprise olanzapine Form II, as disclosed herein.

As used herein, reference to a specified amount or range of amounts of “olanzapine or a pharmaceutically acceptable salt thereof” shall mean that the amount of any pharmaceutically acceptable salt of olanzapine is equivalent to the specified amount or range of amounts of olanzapine.

In some embodiments, the pharmaceutical compositions, syringe comprising the composition and methods as disclosed herein comprise from about 100 mg to about 1,000 mg of olanzapine or a pharmaceutically acceptable salt thereof. For example, the pharmaceutical compositions and methods of the disclosure comprise about 150 to about 700 mg, or about 300 to about 600 mg, of olanzapine or a pharmaceutically acceptable salt thereof. In some embodiments, the pharmaceutical compositions and methods comprise olanzapine Form II, as disclosed herein.

The pharmaceutical compositions and methods of the disclosure provide a therapeutically effective dose of olanzapine for at least 14 days or at least 21 days. In some embodiments, the pharmaceutical compositions and methods provide a therapeutically effective dose of olanzapine for at least about 14 days, 21 days, 28 days, 30 days, 45 days, 56 days 60 days, or 90 days.

According to the described methods, the pharmaceutical compositions can be administered at a frequency of no more than once per month (i.e., no more than once in about 28-30 days). Alternatively, the pharmaceutical compositions can be administered at a frequency of no more than once about every two months (i.e., no more than once in about 56-60 days). In other methods, the pharmaceutical compositions can be administered at a frequency of once per three months (i.e., no more than once in about 84-90 days). A person of ordinary skill in the art would understand references to numbers of days herein to refer to periods of time such that, for example, the expression “for at least about 28 days” would be understood as equivalent to “for a period of at least about 28 days”; and the expression “for at least about 56 days” would be understood as equivalent to “for a period of at least about 56 days.

Further provided is a kit comprising the olanzapine disclosed herein. The kit may comprise a container comprising the olanzapine. The container may be a vial. The vial may include a vial adaptor. The kit may comprise one or more syringe, wherein at least one syringe comprises an excipient, which may be used for reconstitution. The syringe is capable of cooperating with the vial or vial adaptor. The excipient may comprise, for example, a solvent and/or a polymer. The syringe may additionally or alternatively comprise a drug, e.g. a drug different from the API contained in the vial. The syringe may alternatively be empty and serve to withdraw a fluid from a separate container. The syringe in the kit may contain a solvent with which to reconstitute the API. The syringe in the kit may contain a polymer with which to reconstitute the API. The syringe may include both a solvent and a polymer solution with which to reconstitute the API. In some embodiments the solvent is a pharmaceutically acceptable solvent which may be an aqueous solvent or a non-aqueous solvent. Non-limiting examples of solvents include water, benzyl alcohol (BA), benzyl benzoate (BB), dimethyl sulfoxide (DMSO), ethyl acetate, ethyl benzoate, ethyl lactate, ethylene glycol monoethyl ether acetate, glycerol formal, N-methyl-2-pyrrolidone (NMP), triacetin, tributyrin, tripropionin, and mixtures thereof. The vial may contain (e.g. only) olanzapine or a pharmaceutically acceptable salt thereof. In some embodiments the API is olanzapine having at least one of the following properties: a particle size distribution characterized by a D(90) of from about 20 to about 37 μm, a particle size distribution characterized by a D(3,2) of from about 5.5 to about 7.5 μm, or a tapped density from about 0.35 to 0.44 g/ml; or any combination thereof; optionally further comprising a pharmaceutically acceptable excipient, a syringe and/or instructions for use. In some embodiments the olanzapine is olanzapine Form II. The olanzapine may be provided in a container, for example a vial, capsule, bottle, sachet, syringe or ampoule. The kit may optionally further comprise one or more needle or syringe for performing an injection. Said syringe may be operable for reconstituting a solution and or withdrawing a reconstituted solution from the vial. A “reconstituted solution” as used herein, refers to a reconstituted solution or a reconstituted suspension, wherein the API is either fully or partially dissolved. The kit may optionally comprise a syringe adaptor configured to cooperate with the vial or a vial adaptor. The syringe adaptor may for example be configured to be connected to the syringe connection port of a vial adaptor. The syringe may enable fluid mixing. The kit may additionally or alternatively comprise instructions for a user.

For the foregoing embodiments, each embodiment disclosed herein is contemplated as being applicable to each of the other disclosed embodiments. For instance, the elements recited in the method embodiments can be used in the pharmaceutical composition, kit, process and use embodiments described herein and vice versa.

The following examples serve to illustrate the present invention without limiting it.

### EXAMPLES

### Example 1: Standard Methods

### 1. Particle Size Distribution (PSD)

For the purposes herein, particle size distribution (PSD) is determined as the percent volume, i.e. D(90) and D(3,2) and was measured by laser diffraction using a Malvern Mastersizer 2000 equipped with a Hydro 2000 S dispersion unit.

The measurements were performed in saturated water dispersion medium (refractive index=1.22 (general purpose)). The water was saturated with olanzapine to prevent dissolution of the sample while measuring. Dispersion medium was prepared by adding about 3 gm olanzapine to 1 liter water, sonicating in an ultrasound bath for 30 min and filtering (0.22-0.45 μm). The samples were added as concentrated suspension under stirring until an obscuration rate of 10-20% was reached. The results were values arising from ten measurement cycles with a recirculation time (after sonification) of 30 sec. The following parameters were used:
Dispersion unit: Hydro 2000 S
Measuring range: 0.02-2000 μm
Analysis model: General purpose, normal, Mie model
Sensitivity: Normal
Particle shape: Irregular
Sample refractive index: 1.709
Absorption: 0.01
Dispersant: Olanzapine saturated water dispersion medium
Dispersion medium refractive index: 1.330
Obscuration: 10-20%
Pump/stirrer speed: 2500 rpm
Level sensor threshold: 64%
Sample measurement time: 10 s
Background measurement time: 10 s
Repeated measurements (per aliquot): 10
Internal ultrasound: 30 s at 90% power
Recirculation time (after sonication): 30 s
Number of measurements (aliquots): 1

Sample preparation: Samples were prepared as concentrated suspensions. About 50-100 mg of the olanzapine sample was added with a spatula to a small sized glass beaker and several drops of saturated water dispersion medium was added. A paste was obtained by mild mixing, and an additional quantity (1 ml) of saturated water dispersion medium was added in order to obtain a concentrated homogeneous suspension.

Particle size distributions and other powder properties of different batches of olanzapine are given in Table 1.

### 2. Bulk/Tapped Density

Bulk density/tapped density were determined by European Pharmacopoeia 2.9.34. Method 1 (https://www.drugfuture.com/Pharmacopoeia/EP7/DATA/20934E.PDF; PDF downloaded Nov. 14, 2021).

The bulk and tapped densities were determined using the Tapped Density Tester SVM 122 from ERWEKA, equipped with a 100 mL glass cylinder. Approximately 30 g of the powder was carefully poured into the 100 mL measuring cylinder with a funnel. Bulk density was calculated by dividing the amount of powder (in gram) by the measured volume (mL). The tapped density was then determined as follows:

$$Tapp 10 \times - Read volume ( = V 10 )$$

$$Tapp 500 \times ( 10 + 4 \; 90 ) -> Read volume ( = V 500 )$$

$$Tapp 1250 \times ( 500 + 750 ) - Read volume ( = V 1250 )$$

$$Tapp 2500 \times ( 1250 + 12 \; 50 ) - Read volume ( = V 25000 )$$

If the difference between V500 and V1250 was bigger than 2 mL, taps were repeated: i.e. tapped another 1250× and repeated until difference was below 2 mL.
Bulk density=sample weight/volume before tapping.
Tapped density=weight/volume after tapping.
Calculation:

$$Bulk density = m ( mg ) / V 0 ( mL )$$

$$Tapped density = m ( mg ) / V 2500 ( mL )$$

Tapped densities of different batches of olanzapine are provided in Table 1.

### 3. Flow Function Determination

The flow function (FF) was determined by using FT4 Powder Rheometer, Freeman FT4 instrument, and standard Freeman shear test −9 kPa (using small cell 1 ml), according to the procedure below:
Compact to 9 kPa for 60 s
Pre shear hold 7 kPa for 16 s
Shear test at 7 kPa (Shear at 18°/min for 10°, auto detected peak torque yes)
Pre shear hold 6 kPa for 16 s
Shear test at 6 kPa (Shear at 18°/min for 10°, auto detected peak torque yes)
Pre shear hold 5 kPa for 16 s
Shear test at 5 kPa (Shear at 18°/min for 10°, auto detected peak torque yes)
Pre shear hold 4 kPa for 16 s
Shear test at 4 kPa (Shear at 18°/min for 10°, auto detected peak torque yes)
Pre shear hold 3 kPa for 16 s
Shear test at 3 kPa (Shear at 18°/min for 10°, auto detected peak torque yes)

Flowability properties of different batches of olanzapine are provided in Table 1.

### 4. SEM (Scanning Electron Microscope)

Powder samples were fixed on an aluminum stub with conductive double sided adhesive tape and coated with gold. The gold was sputtered using an Edwards S150sputter coater. Samples were scanned on Jeol JSM-5800 scanning microscope, EDS by the Oxford Aztec X-max 20 mm², Conditions: WD=20, HT=10 kV, (mag. 500×, and 1,000×).

### 5. Filling of API in the Vials

Bulk API (i.e. olanzapine Form II described herein) was filled as received into glass vials using semi-automatic powder filling machine using auger technology (SVP 100 by Bausch and Strobel (Germany)). The powder fill machine used sets of augers and agitators to fill the powder in the glass vial. Each vial was subjected to in-process weight control (100%). Vials were closed with rubber caps and aluminum caps and crimped.

### 6. Terminal Sterilization by Dry Heat

Closed and crimped vials filled with API were terminally sterilized at 150-160° C. using dry heat for two hours in dry heat oven SP-470 by Kambič (Slovenia). Additional methods of terminal sterilization include radiation including for example, gamma radiation, X-ray, electron beam and the like with similar results. Alternatively, the API may be prepared aseptically

### 7. JMP Software Analysis

Plots for bivariate fit were prepared and analyzed using JMP® 13.2.1 version software.

### Example 2: Preparation of Micronized Olanzapine Form II

Olanzapine form I (4.00 kg) was loaded into a dissolution vessel and the reactor blanketed with nitrogen. The jacket of the dissolution vessel was heated to 75-80° C. Toluene (48 kg) was loaded into the crystallization vessel and the reactor blanketed with nitrogen. In addition to toluene, acetone, ethyl acetate, n-butyl acetate, or methyl isobutyl ketone may be used as solvent (Sun, et al. Modeling Olanzapine Solution Growth Morphologies. Cryst. Growth Des. 2018, 18, 905-911). The solvent in the crystallization vessel was heated to 75-80° C. The heated toluene was transferred from the crystallization vessel to the dissolution vessel using nitrogen pressure. The mixture was agitated at 75-80° C. until complete dissolution was observed (visually checked). Mechanical filtration of the solution from the dissolution vessel into the crystallization vessel over a heated transfer line and a filter cartridge was performed. The transfer line between the dissolution and crystallization vessels was washed with toluene (0.7 kg). The temperature inside the crystallization vessel was adjusted to 75-80° C. The solution was cooled to 61-63° C. Olanzapine Form II micronized seeds (20 g) were added into the crystallization vessel. The solution was cooled to 53-57° C. over 50-70 minutes using a linear cooling ramp. The solution was further cooled to 43-47° C. over 50-70 minutes using a linear cooling ramp. The solution was then further cooled to 2-8° C. over 80-100 minutes using a linear cooling ramp. The suspension was agitated for 80-100 min at 2-8° C. and then filtered over a filter dryer. The cake was washed with toluene (3.5 kg) cooled to 2-8° C. followed by another wash with toluene (1.7 kg) and an additional cooling to 2-8° C.

The product was dried at a dryer jacket temperature of 75-100° C. and under vacuum of 100-200 mbars until a limit of detection (LOD) level below 0.5% was achieved after which the dry material was cooled to 20-30° C. and unloaded from the filter dryer. The dry material was sieved using a vibratory sifter over a 300 micron screen size. The sieved material was then micronized on an air-jet micronizer using nitrogen pressure ranging from 1.5-4 bars. The XRD profile of olanzapine Form II as described here is shown in FIG. 1. Main typical peaks of olanzapine Form II are defined as peaks at about 8.7, 12.5, 17.4, 19.9, 21.1, 21.6, 22.4, 24.0, 25.3 and 29.8±0.2 deg. 2θ. The particle morphology of the olanzapine Form II disclosed herein is irregular and is substantially as shown in FIG. 2 (×1,000 magnification).

### Example 3: Preparation of Sterile Vials with Olanzapine

Each of the batches from Example 1 was filled in vials by auger filling and terminally sterilized according to Example 1, sections 5 and 6. During each filling run, the critical parameters of filling performance were monitored including relative standard deviation (RSD) of fill weight (%) and filling speed (% of the maximum capacity of the machine according to its technical specification).

Filling performance was measured by two parameters: RSD of fill weight and filling speed. RSD of fill weight is a direct measure of the filling operation accuracy, where a smaller number refers to a lower variation. Fill weight variation influences the uniformity of dose in the final drug product, which is a critical quality attribute. Filling speed determines production time and a higher speed is preferred. Results of the filling performance parameters for each API batch are shown in Table 1.

During initial filling experiments of micronized olanzapine in the vials, significant differences in filling performance between different batches was noted. The filling was rated as very poor to good filling.

### Results

Without limitation, RSD of filling weight is no more than about 2.0%, specifically 1.5% or less, more specifically about 1.0% to about 1.5%.

Without limitation, in some embodiments, filling speed is about 14 to about 20% and may differ depending on capacity of the filling machine according to its technical specification. In some embodiments filling of container e.g. vialling, is performed by auger filling. In some embodiments, filling of container e.g. vialling, is performed by vacuum filling. The filling method is not limiting as methods of filling may be contemplated. In order to improve filling properties of olanzapine, and quantify filling performance of different production batches, olanzapine properties such as particle size distribution by LALLS (PSD), tapped density (TD), and flow function (FF) by Freeman, were analyzed relative to filling performance: relative standard deviation (RSD) of filling weight, and filling speed (% of the maximum capacity of the machine according to its technical specification), Table 1.

TABLE 1
Comparison of olanzapine properties vs. process
parameter/vials filling performance

| | D(90) | D(3,2) | TD | Flow | RSD of fill | Filling |
|---|---|---|---|---|---|---|
| Batch | (μm) | (μm) | (g/mL) | function | weight (%) | speed (%) |
| M1-010 | 13 | 4.2 | 0.37 | 1.4 | 0.77 | 14 |
| M6-020 | 24 | 6.6 | 0.41 | 2.0 | 1.24 | 16 |
| M7-121 | 42 | 8.3 | 0.50 | 2.1 | 1.62 | 18 |
| M2-121 | 30 | 6.4 | 0.39 | 1.5 | 1.49 | 14 |
| S1-920 | 11 | 4.1 | 0.29 | 1.2 | 0.91 | 8 |
| S2-920 | 38 | 7.6 | 0.45 | 2.0 | 1.96 | 20 |
| S3-020 | 17 | 5.4 | 0.28 | 1.3 | 0.88 | 12 |
| S1-220 | 26 | 6.1 | 0.39 | 1.5 | 1.0 | 14 |
| S3-221 | 26 | 6.4 | 0.40 | 1.8 | 1.34 | 18 |

Results presented in Table 1 show that the most indicative parameter for desired flow and filling properties and subsequent reconstitution and injectability is D(90) particle size, D(3,2) or tapped density.

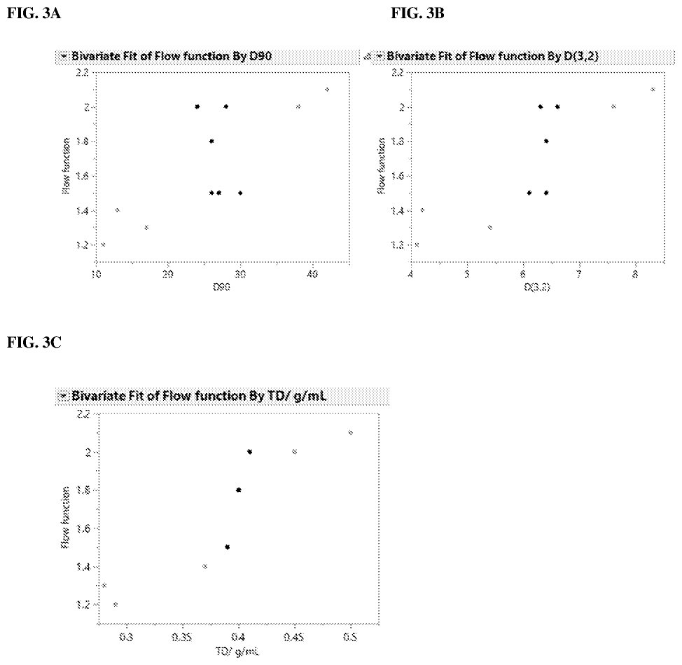

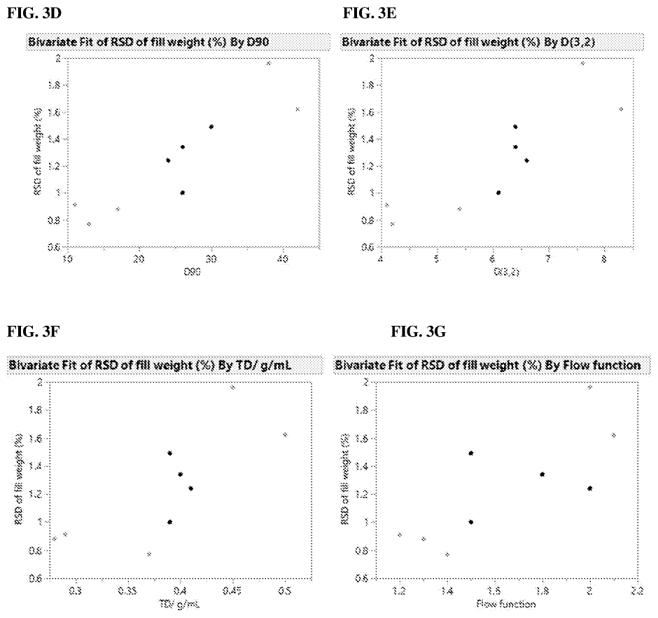

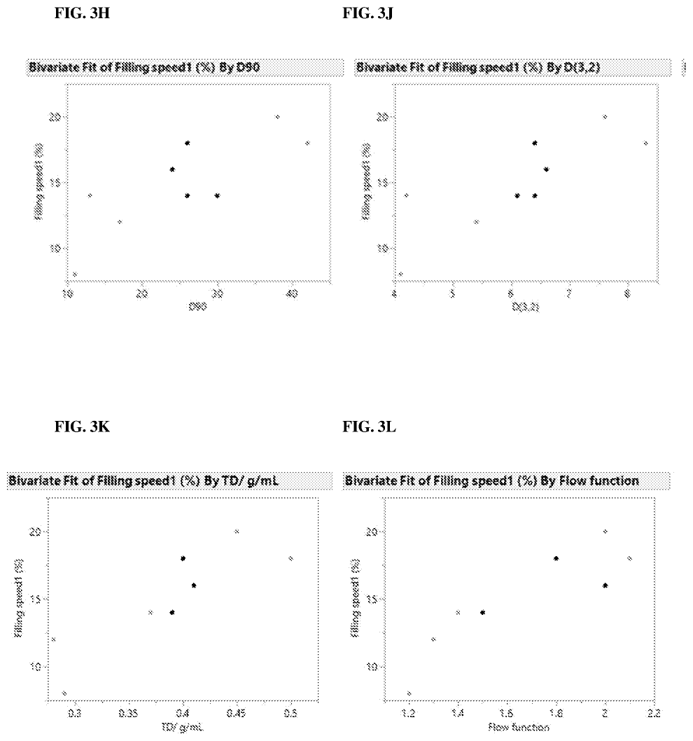

Additionally, analysis was done in JMP software to correlate the parameters of API with filling performance, and the parameter shown to be most significant is particle size at D(90), D(3,2), or tapped density. This analysis may be visualized in FIGS. 3A-3L. The clustered dark circles in the center of the plots represent the preferred values for each feature. All the batches having a desired flow and fill are depicted by the dark circles. All the dark circles are clustered in the bivariate fit analyses. When all plots are shown together with batches that fulfil criteria all black circles fall within the ranges as described.

Bivariate fit of flow function to the variables, D(90), D(3,2) and TD are shown in FIGS. 3A-3C respectively. In FIG. 3A the clustered dark circles show that for D(90) from about 20 to about 37 μm the flow function is equal to or greater than 1.4, specifically from about 1.4 to about 2.0. In FIG. 3B, the clustered dark circles show that for D(3,2) from about 5.5 to about 7.5 μm, the flow function is below 2.0, specifically from about 1.4 to about 2.0. In FIG. 3C, the clustered dark circles show that TD from about 0.38 g/mL to about 0.44 the flow function is from about 1.4 to about 2.0. In some embodiments the flow function is from about 1.5 to about 2.0.

Bivariate fit of RSD of fill weight (%) by the variables, D(90), D(3,2), TD and flow function are shown in FIGS. 3D-3G respectively. In FIG. 3D the clustered dark circles show that for D(90) from about 20 to about 37 μm the RSD of fill weight (%) is below 1.5%. In FIG. 3E the clustered dark circles show that for D(3,2) from about 5.5 to about 7.5 μm the RSD of fill weight (%) is below 1.5%. In FIG. 3F the clustered dark circles show that for TD from about 0.35 to about 0.44 g/mL the RSD of fill weight (%) is below 1.5%. In FIG. 3G the clustered dark circles show that at a flow function below 2.0, specifically from about 1.4 to about 2.0 the RSD of fill weight (%) is below 1.5%.

Bivariate fit of filling speed by the variables, D(90), D(3,2), TD and flow function are shown in FIGS. 3H-3L respectively. In FIG. 3H the clustered dark circles show that for D(90) from about 20 to about 37 μm the filling speed is 14% or higher. In FIG. 3J the clustered dark circles show that for D(3,2) from about 5.5 to about 7.5 μm the filling speed is 14% or higher. In FIG. 3K the clustered dark circles show that for TD from about 0.35 to about 0.44 g/mL the filling speed is 14% or higher. In FIG. 3L the clustered dark circles show that at a flow function below 2.0, specifically from about 1.4 to about 2.0 the filling speed is 14% or higher.

TABLE 2
Olanzapine parameters with good flow and filling performance

| | | | | | RSD (%) | Filling |
|---|---|---|---|---|---|---|
| | PSD (μm) | | TD | Flow | filling | speed |
| | D(90) | D(3,2) | (g/mL) | function | weight | (%) |
| Parameter | 20 to | 5.5 to | 0.35 to | 2.0 or | 1.5 or | ≥14 |
| range | 37 | 7.5 | 0.44 | less | less | |
| further | 24 to | 5.8 to | 0.37 to | 1.4 to | 1.0-1.5 | 14-20 |
| parameter | 35 | 7.0 | 0.41 | 2.0 | | or >20 |
| ranges | | | | | | |

The manufacturing process for the final drug product includes a terminal sterilization step, and specifically the quality of the bulk API should not be affected by sterilization. Therefore, after the dry heat sterilization, samples of API in vials filled with two batches (with low PSD) were analyzed for PSD and compared to starting API material. Results are shown in Table 3.

TABLE 3
PSD of bulk API compared to PSD of API
in vial after dry heat sterilization

| | D(90) | D(3, 2) |
|---|---|---|
| Sample Name | (μm) | (μm) |
| S1-920 | 11 | 3.9 |
| Sterilized vial with S1-920 | 13 | 5.4 |
| S3-020 | 16 | 5.2 |
| Sterilized vial with S3-020 | 16 | 5.8 |
| S1-220* | 29 | 5.8 |
| Sterilized vial with S1-220 | 28 | 7.0 |
| M1-010 | 14 | 4.7 |
| Sterilized vial with M1-010 | 14 | 5.2 |
| M6-020 | 24 | 6.5 |
| Sterilized vial with M6-020 | 22 | 7.1 |
| M2-121 | 40 | 7.5 |
| Sterilized vial with M2-121 | 44 | 8.9 |

*Average from three drums of a single batch

Table 3 shows that pre-dry heat sterilization olanzapine drug substance having a PSD characterized by a D(90) of 20-37 μm undergoes slight change after the terminal sterilization process and D(90) remains within the acceptable range. Table 3 also shows that pre-dry heat sterilization olanzapine drug substance having a PSD characterized by a D(3,2) of 5.5-7.5 μm undergoes slight change after the terminal sterilization process and D(3,2) remains within the acceptable range.

There may be a desire to control the size of the largest particles, D(99) and D(100), for example when the final product is a solution or suspension for parenteral administration. In some embodiments olanzapine PSD characterized by D(99) and D(100) are limited to about ⅔, ½ or about ⅓ of the internal diameter (I.D.) of the dosing needle. Without wishing to be bound to theory, a value above this value may increase the risk of needle blockages and syringeability issues. For example, a 21 gauge (21 g) needle has an I.D. of about 490-510 μm. Some typical D(90), D(99) and D(100) values are shown in Table 4.

TABLE 4
D(90), D(99) and D(100) parameters

| Sample Name | D(90)/μm | D(99)/μm | D(100)/μm |
|---|---|---|---|
| Olanzapine S1-920 | 11 | 16 | 21 |
| Olanzapine S2-920 | 38 | 86 | 136 |
| Olanzapine S3-020 | 16 | 27 | 90 |
| Olanzapine S1-220 | 29 | 83 | 142 |
| Olanzapine S2-121 | 27 | 91 | 196 |
| Olanzapine S3-221 | 24 | 42 | 61 |
| Olanzapine S4-221 | 26 | 64 | 110 |
| Olanzapine M1-010 | 14 | 25 | 42 |
| Olanzapine M6-020 | 24 | 84 | 205 |
| Olanzapine M7-121 | 40 | 106 | 205 |

Furthermore, results of PSD parameter D(100) obtained by stratified sampling using a sampling thief of the batches show that the particles have a PSD characterized by D(99) less than about 100 μm and/or a PSD characterized by D(100) less than about 250 μm, less than about 210 μm or from about 60 to about 210 μm.

The upper limit of the preferred D(90) range may be also limited with regard to presence of largest particle as measured by D(100). Specifically, the olanzapine Form II as described herein is characterized by a D(100) less than the inner diameter (I.D.) of the administration needle. For example a standard 21 g, 16 mm long needle is approximately 0.49-0.51 mm.

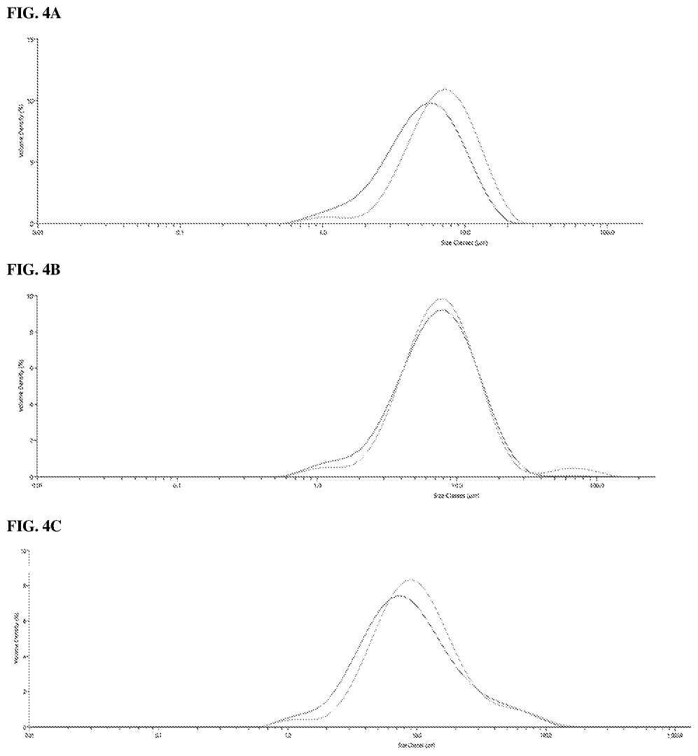

FIGS. 4A-4C show PSD of bulk API compared to PSD of API in vial after sterilization. FIG. 4A: Olanzapine API S1-920 (slightly left most curve), API in vial after sterilization (right most curve). FIG. 4B: Olanzapine API S3-020 before (lower curve) and after sterilization (upper curve) shows curves are highly similar. FIG. 4C: Olanzapine API S1-220 (slightly left most curve), API in vial after sterilization (right and slightly higher curve).

All patent documents and publications are herein incorporated by reference to the same extent as if each individual publication was specifically and individually incorporated by reference. The invention illustratively described herein suitably may be practiced in the absence of any element(s) not specifically disclosed herein. Thus, for example, in each instance herein any of the terms “comprising”, and “consisting of” may be replaced with either of the other two terms. For the foregoing embodiments, each embodiment disclosed herein is contemplated as being applicable to each of the other disclosed embodiments. For instance, the elements recited in the API embodiments can be used in the pharmaceutical composition, kit and method embodiments described herein and vice versa. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be contemplated by those skilled in the art, and that such modifications and variations are considered to be within the scope of this invention as defined by the appended claims.

1. Olanzapine, or pharmaceutically acceptable salt thereof, having a particle size distribution characterized by a D(90) of from about 20 μm to about 37 μm, a particle size distribution characterized by a D(3,2) of from about 5.5 μm to about 7.5 μm, or a tapped density from about 0.35 g/ml to 0.44 g/ml, or any combination thereof.

2. The olanzapine of claim 1, wherein the particle size distribution is characterized by a D(90) of from 20 μm to about 37 μm.

3. The olanzapine of claim 2 wherein the particle size distribution is characterized by a D(90) of from about 24 μm to about 35 μm.

4. The olanzapine of claim 1, wherein the particle size distribution is characterized by a D(3,2) of from about 5.5 μm to about 7.5 μm.

5. The olanzapine of claim 4, wherein the particle size distribution is characterized by a D(3,2) of from about 5.8 μm to about 7.0 μm.

6. The olanzapine of claim 1, wherein the tapped density of the olanzapine is from about 0.35 g/ml to about 0.44 g/ml.

7-10. (canceled)

11. The olanzapine of claim 1, wherein the flow function of the olanzapine, as measured by a FT4 Powder Rheometer, is about 2.0 or less.

12. (canceled)

13. The olanzapine of claim 1, wherein the wherein the RSD % of fill weight is 1.5% or less, and/or wherein the filling speed is 14% or higher.

14. The olanzapine of claim 1, wherein the olanzapine is olanzapine Form II.

15. A container comprising the olanzapine of claim 1.

16. The container of claim 15, wherein the container is a vial, a syringe or an ampoule.

17-20. (canceled)

21. A pharmaceutical composition comprising the olanzapine of claim 1, and one or more pharmaceutically acceptable excipient.

22. The pharmaceutical composition of claim 21, wherein the pharmaceutical composition is in the form of a solution or suspension for parenteral administration.

23. The pharmaceutical composition of claim 21, in the form of an aqueous or non-aqueous suspension.

24. The pharmaceutical composition of claim 21, wherein the one or more pharmaceutically acceptable excipient comprises a polymer or non-polymer.

25. The pharmaceutical composition of claim 24, wherein the excipient comprises a biodegradable polymer and the comprises poly(lactide), poly(glycolide), poly(lactide-co-glycolide), poly-1-lactic acid, poly-d-lactic acid, polyethylene glycol or a copolymer of the foregoing, poly(aliphatic carboxylic acids), copolyoxalates, polycaprolactone, polydioxanone, poly(ortho carbonates), poly(acetals), poly(lactic acid-caprolactone), polyorthoesters, poly(glycolic acid-caprolactone), poly(amino acid), polyesteramide, polyanhydrides, polyphosphazines, poly(alkylene alkylate), biodegradable polyurethane, polyvinylpyrrolidone, polyalkanoic acid, albumin, chitosan, casein, waxes or blends or copolymers thereof.

26-32. (canceled)

33. A kit comprising the olanzapine of claim 1; optionally further comprising a pharmaceutically acceptable excipient, a syringe and/or instructions for use.

34. A method of treating a subject suffering from a neuropsychiatric disease or otherwise in need of the treatment, comprising administering to the subject the olanzapine of claim 1, wherein the olanzapine provides a therapeutically effective plasma concentration of olanzapine for a period of at least about 14 days or at least 21 days after administration to the subject.

35-39. (canceled)

40. A process of filling a container with olanzapine or a pharmaceutically acceptable salt thereof, the process comprising providing olanzapine having a particle size distribution characterized by a D(90) of from about 20 μm to about 37 μm, a particle size distribution characterized by a D(3,2) of from about 5.5 μm to about 7.5 μm, or a tapped density from about 0.35 g/ml to 0.44 g/ml; or any combination thereof; and
filling the container using a semi-automatic or fully automatic filling machine;
thereby filling the container with the olanzapine or a pharmaceutically acceptable salt thereof.

41. A process of filling a container with olanzapine or a pharmaceutically acceptable salt thereof, wherein the RSD % of the filling weight is less than 1.5% or the filling speed is 14% or higher,
thereby filling the container with the olanzapine or a pharmaceutically acceptable salt thereof.

42-45. (canceled)
